(19) United States
(12) Patent Application Publication
(10) Pub. No.: US 2013/0259844 A1
(43) Pub. Date: Oct. 3, 2013

(54) Fermented Foodstuff

(71) Applicants: Peter Brooks, Newton Abbot (GB); Jane Beal, Plymouth (GB); Soumela Savvidou, Komotini (GR); Vlasta Demeckova, Kosice (SK)

(72) Inventors: Peter Brooks, Newton Abbot (GB); Jane Beal, Plymouth (GB); Soumela Savvidou, Komotini (GR); Vlasta Demeckova, Kosice (SK)

(73) Assignee: University of Plymouth, Plymouth (GB)

(21) Appl. No.: 13/876,386

(22) PCT Filed: Sep. 29, 2011
(86) PCT No.: PCT/GB2011/001426

§ 371 Date: Jun. 12, 2013

(30) Foreign Application Priority Data
Sep. 30, 2010 (GB) 1016456.4

(51) Int. Cl. A23L 1/30; C12R 1/225; C12R 1/25; C12N 1/20

(52) U.S. Cl. CPC A23L 1/3014; C12N 1/20; C12R 1/225; C12R 1/25
USPC 424/93.45; 435/252.9

(57) ABSTRACT

A fermented foodstuff composition for human consumption is provided, the foodstuff composition being prepared by the fermentation of a foodstuff substrate with a lactic acid producing bacteria, the lactic acid bacteria being characterised by:
a) being viable under the conditions prevailing in the human gastrointestinal tract;
b) being a bacteria capable of aggregating and/or coaggregating with one or more pathogens; and
c) being able to produce upon fermentation in the foodstuff substrate lactic acid in an amount of at least a minimum inhibitory concentration of lactic acid.

The present invention relates to foodstuffs for humans, in particular fermented foodstuffs, and to methods for their preparation and the use thereof. The invention is especially concerned with the provision of functional foodstuffs for humans, that is foodstuffs that provide benefits to the consumer beyond the nutritional value of the food, for example increasing gut motility, satiety, and the like.

It is known to prepare foodstuffs for animals by the fermentation of a suitable substrate with bacteria. The process of producing fermented feed can be seen as a form of ‘biopreservation’. EP 0 906 952 discloses a bacterial strain for the ensiling of straw fodder. The strain, of the genus Lactococcus, was found to be effective in inhibiting the growth of yeast, clostridia, mould, gram positive bacteria and certain gram negative bacteria in the ensiling of green fodder.

Further, US 2002/0054935 is concerned with a livestock feed composition suitable for the fattening of livestock, such as cattle, goats, sheep, swine and fowl. The nutritional value of the livestock feed is increased by inoculation with one or more strains of Aspergillus. The livestock feed treated in this way consists of a fibrous feed material, a cereal, and an organic waste material. It appears that the Aspergillus is used to modify the nutrient content of the feed. However, this can have disadvantageous results, as many Aspergillus spp. produce mycotoxins harmful to many animals.

U.S. Pat. No. 6,403,084 is concerned with mixed cultures for improved fermentation and aerobic stability of silage. The problem of aerobic instability of silage is addressed, in particular the rapid growth of yeast and mould that can occur, resulting in the silage being spoiled. Further, it is noted that silage may be spoiled by the growth of yeast, even when inoculated and subjected to a good fermentation phase, in which microorganisms are used to ferment the silage and produce lactic acid, reducing the pH and giving rise to acid conditions. Acid-tolerant yeasts are considered to be responsible for the spoilage of fermented silage. As a solution to these problems, U.S. Pat. No. 6,403,084 proposes inoculating the silage with a combination of the homofermentative lactic acid bacteria Lactobacillus plantarum and the heterofermentative lactic acid bacteria Lactobacillus buchneri or Lactobacillus brevis. The aforementioned combination of microorganisms is alleged to provide sufficiently low pH conditions to preserve the silage and prevent spoiling due to the growth of mould and yeast.

WO 99/18188 describes a feed product for horses. The feed product comprises one or more strains of Lactobacillus having the ability to colonize the equine intestines. The microorganisms were isolated from the gastric or intestinal mucosa of horses.

GB 2,167,639 discloses a process for the treatment of industrial or agricultural waste matter, such as animal protein. The process involves chopping the waste as an aqueous mass and treating the resulting material with proteolytic enzymes to form a suspension, obtaining a gelatinised starch content in the suspension and adding to the suspension amylolytic enzymes and a lactic acid producing culture. The resulting mixture is fermented to produce simple sugars and lactic acid.

U.S. Pat. No. 4,214,985 relates to sewage treatment. The treatment involves inoculating sewage sludge with L. plantarum bacteria and a carbohydrate, such as lactose. The resulting mixture is fermented until the pH falls below 4.0. The thus produced composition is used as a soil extender.

JP 2007082468 is concerned with providing a microorganism preparation for feed. The preparation comprises particular strains of Lactobacillus plantarum and/or Bacillus subtilis. Fermented feeds may be produced by adding the microorganisms to organic wastes, such as silage grass, and fermenting.

WO 89/05849 describes the selection of lactic acid bacteria isolated from the gastrointestinal tract of pigs for their ability to survive in the environment of the gastrointestinal tract and to adhere to the epithelium of the gastrointestinal tract of the target animal. The bacteria selected with these properties may be included in a fermented milk product for human consumption or in a veterinary composition for providing to pigs for the prevention or treatment of gastrointestinal diseases.

More recently, WO 2008/006382 discloses homofermented liquid animal feed products. As discussed in WO 2008/006382, the production of fermented animal feeds using microorganism-containing inoculants is very difficult, often leading to the fermented feed containing pathogenic bacteria, such as Vibrio spp., Campylobacter spp., Salmonella spp., E. coli, and Staphylococcus aureus. The fermented feed may also contain a high content of various yeasts and moulds. It is noted that the ingestion by the livestock of such inappropriately fermented feeds may result in morbidity and mortality. WO 2008/006382 notes that the sterile handling of bacteria required by farmers wishing to prepare their own fermented feed is often impossible to achieve. Further, there is a practice of using a continuous fermentation process, in which a portion of one batch of fermented feed is used as an inoculum for a subsequent fermentation batch. This leads to a gradual build up of harmful and undesirable microorganisms in the fermented feed. In an attempt to address these problems, WO 2008/006382 proposes a method for preparing a fermented mixed feed, the method comprising: providing a liquid fermented product; providing a feed product to be fermented; combining the aforementioned products; and fermenting the feed product using the liquid fermented product as an inoculum. A fermented feed prepared by this method is also described.

EP 0 955 061 addresses the issue of gastroenteric infections in pigs, in particular porcine rotavirus, porcine coronavirus, enterotoxigenic and enteropathogenic strains of Escherichia coli, Clostridium sp., Salmonella sp., Serpulina hyodysenteriae, Serpulina pilosicoli, Lawsonia intracellularis, Isospora suis, and Cryptosporidium. As a solution to the problem of gastroenteric infections in pigs, EP 0 955 061 proposes an oral product characterised by containing at least one specific antibody to the aforementioned microorganisms, derived from the egg yolks of immunized hens. It is noted in EP 0 955 061 that lactacidogenic bacteria administered to pigs can have a probiotic effect, suppressing the propagation of the enteropathogenic or enterotoxigenic bacteria and enhance the activity of the animal's immune system. Accordingly, a preferred embodiment of EP 0 955 061 includes one or more lactic acid bacteria, such as Enterococcus spp. and Lactobacillus spp.

As discussed in EP 0 955 061, young animals are particularly susceptible to infections of the GI tract, leading to illness and death. Ways of improving the health and wellbeing of finishing pigs are described by P. Brooks et al., ‘The Effect on Biological Performance and Faecal Microbiology of Feeding Finishing Pigs on Liquid Diets Fermented with Lactic Acid Bacteria’, SafePork, 2005, page 149. Brooks et al. note that fermented liquid feeds (FLF) have been shown to reduce the incidence of salmonella in pigs. In particular, it was found that a lactic acid concentration of 70 mMol/kg in the fermented feed exhibited bacteriostatic activity with respect to Salmonella spp., but higher concentrations of lactic acid in excess of 100 mMol/kg were needed to be bactericidal. However, Brooks et al. had found that natural fermentations had produced unpredictable results in commercial feed units and referred to a study that found that only 3% of commercial fermentations of wheat and barley produced more than 75 mMol/kg of lactic acid after 24 hours of fermentation. It was concluded that fermentations to produce lactic acid in high concentrations relying on indigenous microorganisms present in the grains could not be relied upon for commercial production of fermented feeds. Brooks et al. conducted experiments using specific LAB to examine the effect on biological performance and faecal microbiology of pigs fed diets of fermented liquid feeds. The results showed that the pigs retained good health when fed on the fermented liquid feed, showing no change in average daily weight gain when fed with the FLF compared with a standard feed. In addition, the experiments showed that, while the fermented feed contained lactic acid bacteria in high concentrations, the concentration of LAB in the faeces of the pigs remained unchanged. However, analysis of the faeces for the presence of coliforms indicated that the coliform content was reduced in the pigs fed with the FLF diet. This in turn indicated an improvement in the health of the pig and a lower risk of infection and illness. It was concluded that the selection of the LAB used for fermentation was important in achieving the reduction in coliforms.

As noted by Brooks et al., achieving a specific concentration of lactic acid in the fermented feed is important in achieving the beneficial effects of the fermented feeds. Techniques for measuring the concentrations of lactic acid in fermented feeds are described by S. J. Niven, et al., ‘The Simultaneous Determination of Short Chain Fatty Acid, Monosaccharides and Ethanol in Fermented Liquid Pig Diets’, Animal Feed Science and Technology, 117 (2004), pages 339 to 345.

The thesis of V. Demeckova, ‘Benefits of Fermented Liquid Diets for Sows and their Piglets’, Department of Agriculture and Food, Faculty of Land, Food and Leisure, University of Plymouth, July 2003, describes experiments conducted with liquid feed fermented with Lactobacillus plantarum to determine their effects on the antimicrobial and potential immunological effects on sows in late gestation periods. The results indicated that certain strains of Lactobacillus were both an effective inoculant for the preparation of fermented liquid feeds, as well as providing probiotic activity to the sow once the fermented feed was ingested. Significantly, immunoglobulin levels in the sows' colostrums were increased. Colostrum from sows fed fermented feed also increased the mitogenic activity of blood lymphocytes and enterocytes. These factors could in turn improve the resistance of the sows and their piglets to pathogen challenges.

WO 2005/007834 discloses an acid tolerant probiotic Lactobacillus plantarum Probiotic-38 that can suppress the growth of certain pathogens in a host, while being tolerant to gastric and bile acids.

Most recently, copending unpublished international patent application PCT/GB2010/000650 concerns the provisions of fermented feeds for animals using lactic acid producing bacteria (LAB) having certain characteristics. The feeds thus produced were shown to have significant positive effect in the rearing of young animals, in particular poultry. The lactic acid producing bacteria used in preparing the fermented animal feed were characterised by:
a) being viable under the conditions prevailing in the gastrointestinal tract of the target animal;
b) being a bacteria capable of aggregating and/or coaggregating with one or more pathogens; and
c) being able to produce upon fermentation in the feed substrate lactic acid in an amount of at least a minimum inhibitory concentration of lactic acid.

While the advantages of providing fermented feed to animals has been shown, it would be beneficial if a similarly advantageous foodstuff could be provided for human consumption. It would be particularly advantageous if the foodstuff could exhibit a probiotic effect for the persons consuming it and increase their resistance to infections. In particular, it would be most beneficial if the foodstuff could provide resistance to infections of such organisms as Salmonell spp., Escherichia coli, strains of MRSA, and Clostridium difficile.

Drago, L., et al., ‘Inhibition of in vitro growth of enteropathogens by new Lactobacillus isolates of human intestinal origin’, FEMS Microbiology Letters, 153 (1997), pages 455 to 463, describe experiments to isolate strains of Lactobacillus from the faeces of new born infant humans and examine their effect in co-cultures on certain pathogenic bacteria. The experiments conducted were entirely in vitro and, while showing some beneficial effects of the Lactobacillus strains in reducing the growth of certain pathogens, did not relate at all to the formulation of feeds.

Fermented foodstuffs for human consumption are known in the art. For example, Yakult® is a commercially available foodstuff prepared from the fermentation of milk with Lactobacillus casei. The fermented foodstuff had the Lactobacillus casei microorganisms present in a concentration of up to 10 billion per 100 ml.

EP 1 884 566 discloses a lactic acid bacteria fermentation product containing viable lactic acid bacteria at high concentrations and to foodstuffs, in particular fermented milk products, containing the lactic acid bacteria fermentation product. The foodstuffs are intended for human consumption.

EP 1 661 983 concerns certain lactic acid producing bacteria and compositions comprising the same. The bacteria are indicated to be capable of stimulating mucosal immunity in a host. The compositions may be in the form of foods, beverages or pharmaceutical products.

The inventors have now found that a particularly advantageous foodstuff for human consumption may be prepared by the fermentation of a suitable substrate using one or more lactic acid bacteria possessing certain characteristics.

Accordingly, in a first aspect, the present invention provides a fermented foodstuff composition for human consumption, the foodstuff composition being prepared by the fermentation of a foodstuff substrate with a lactic acid producing bacteria, the lactic acid bacteria being characterised by:
a) being viable under the conditions prevailing in the human gastrointestinal tract;
b) being a bacteria capable of aggregating and/or coaggregating with one or more pathogens; and
c) being able to produce upon fermentation in the foodstuff substrate lactic acid in an amount of at least a minimum inhibitory concentration of lactic acid.

It has been found that a fermented foodstuff according to the present invention produced by the fermentation of a food substrate with a lactic acid producing bacteria having the characteristics set out above provides significant advantages over known foods. In particular, the foodstuff is able to be stored for extended periods of time and be transported without spoiling, the fermented food being resistant to the growth of mould and yeasts and resistant to invasion and colonisation by bacteria potentially harmful to humans. Further, the fermented foodstuff, once consumed, provides significant protection for the person against infection by bacteria, in particular pathogenic bacteria likely to cause serious illness or even death of the person. The fermented foodstuff has been demonstrated to have particular activity against such organisms as Salmonell spp., Escherichia coli, strains of MRSA and Clostridium spp., which pose a significant threat to humans, in particular the young, the elderly and the infirm.

The foodstuff is prepared by the fermentation of a suitable substrate, which is inoculated with a culture containing the lactic acid producing bacteria and fermented. The thus inoculated and fermented substrate may itself form the finished foodstuff. Alternatively, the substrate, once fermented may be added to other food materials, in order to provide the other materials with the probiotic and immune stimulatory effects.

The substrate may be any suitable substrate that may be consumed by humans in a fermented condition. The substrate may consist of a substrate from a single source or, alternatively may comprise a combination of substrates.

Suitable substrates include organic materials, such as milk and milk fractions, for example skimmed milk and whew, raw or cooked cereals and/or cereal fractions, for example oats, wheat, barley, maize, millet and sorghum. Further suitable substrates include other carbohydrate-rich food sources, for example potato starch and cassaya. Oats and partially cooked oats are a particularly suitable substrate. The substrate may be supplemented with other components, such as minerals and vitamins, depending upon the composition of the substrate, to meet the nutritional requirements of the target persons, for example when used as a therapeutic food in the case of the infirm, frail or elderly, or as a weaning food, in the case of infants.

To be suitable for fermentation, the substrate should contain water in an amount sufficient to support fermentation with the lactic acid producing bacteria. Preferably, the substrate has a water content of at least 20% by weight, more preferably at least 30% by weight, still more preferably at least 40% by weight. Preferred ratios of dry substrate to water are dependent on the substrate and range from 1:0.25 to 1:4, more preferably from 1:0.4 to 1:2.

The foodstuff substrate may contain sufficient water to support fermentation with the lactic acid bacteria. If not, water should be added to the foodstuff substrate to achieve the water content required for fermentation. The water quality should be sufficient for the target persons.

To produce the fermented foodstuff of the present invention, the substrate is inoculated with lactic acid producing bacteria and fermented. The lactic acid producing bacteria employed in the present invention are characterised by the following features:
a) The inoculant bacteria are viable under the conditions prevailing in the human gastrointestinal tract;
b) The bacteria are capable of aggregating and/or co-aggregating with one or more pathogens; and
c) The bacteria are capable of producing lactic acid upon fermentation with the foodstuff substrate to at least a minimum inhibitory concentration in the fermented foodstuff.

Bacteria having the three characteristics (a) to (c) give rise to the advantageous properties of the fermented foodstuff of the present invention. As noted above, the foodstuff substrate is inoculated with the lactic acid producing bacteria.

Accordingly, in a further aspect, the present invention provides an inoculant for the preparation of a fermented human foodstuff from a foodstuff substrate, the inoculant comprising a viable culture of a lactic acid producing bacteria having the following characteristics:
a) being viable under the conditions prevailing in the human gastrointestinal tract;
b) being a bacteria capable of aggregating and/or co-aggregating with one or more pathogens; and
c) being able to produce upon fermentation in the foodstuff substrate lactic acid in an amount of at least a minimum inhibitory concentration of lactic acid.

The inoculant may be in any suitable form and of any suitable composition so as to contain viable lactic acid producing bacteria for populating and fermenting the foodstuff substrate and populating the gastrointestinal tract (GTI) of the consumer. Preferred presentations for the inoculant are freeze dried or as a liquid culture.

The inoculant should contain the lactic acid producing bacteria in a viable form and in sufficient concentration to allow the foodstuff substrate, once inoculated, to ferment and produce the required number of viable lactic acid producing bacteria and the required concentration of lactic acid in the fermented product. A typical number of lactic acid producing bacteria in the inoculant is from 10⁵ to 10⁹ CFU/ml, more preferably about 10⁶ CFU/ml, if presented in liquid form or 10⁵ to 10⁹ CFU/g, more preferably about 10⁶ CFU/g if presented in freeze dried form.

For example, a suitable inoculant for a substrate is 0.1% of a liquid broth culture containing 10⁹CFU/ml of the lactic acid producing bacteria or 0.1% of a freeze dried culture containing 10⁹ CFU/g of the lactic acid producing bacteria.

The inoculant organism may be presented in a suitable carrier to maintain shelf life and facilitate accurate dispersion when added to the foodstuff substrate. Such methods are known in the art and readily understood by the person skilled in the art.

As a first characteristic, the lactic acid producing bacteria should be viable and survive in the human gastrointestinal tract (GIT). The conditions in the human gastrointestinal tract prevent the colonisation and growth of many microorganisms and potential enteropathogens. However, the microflora of the gut may be perturbed by ill-health or the administration of medications, such as antibiotics, rendering the GIT more susceptible to enteropathogens. If the subject is immune compromised, as in the case of HIV infections, susceptibility to such pathogens is further increased. In order to provide the advantageous properties of the fermented foodstuff of the present invention, the lactic acid producing bacteria used to ferment the foodstuff substrate should be viable under the acidic conditions prevailing in the human upper gastrointestinal tract and the alkaline conditions encountered in the human duodenum. Further, the lactic acid producing bacteria should remain viable in both the small intestines and the large intestines.

The viability of the bacteria in the gastrointestinal tract may be determined by methods and techniques known in the art. In particular, the microbial count of the viable lactic acid bacteria in the faeces of a person fed a diet containing viable lactic acid bacteria may be measured. Such methods are known in the art and readily understood by the person skilled in the art. For example, it is known to produce an ELISA or a 16nRNA assay specific to the microorganisms being assessed.

As a further alternative or in addition thereto, the viability of the lactic acid producing bacteria in the human gastrointestinal tract may be determined in vitro, in particular by measuring the growth of the microorganisms under acidic and alkaline conditions similar to or the same as those prevailing in the human stomach and duodenum. Thus, the viability of the lactic acid producing bacteria may be determined after exposure to pH 2 for 2 hours followed by buffering to pH 6.0 to 8.0 for 4 hours in a suitable foodstuff substrate, to represent conditions in the stomach and the duodenum of a person.

In order to more accurately model the conditions of the human gastrointestinal tract, it is further preferred that the in vitro experiments described hereinbefore are conducted using the foodstuff substrate of the eventual fermented foodstuff composition as the growth medium for the microorganisms, as this may have a buffering effect and influence pH. In this way, any effects produced within the gastrointestinal tract when a person consumes the fermented foodstuff that may alter the conditions therein and/or the viability of the lactic acid producing bacteria may be determined.

A procedure for the in vitro determination of the viability of a lactic acid producing bacteria in the human gastrointestinal tract is described in detail in Example 1 hereafter.

In a particularly preferred embodiment, the fermented foodstuff of the present invention comprises lactic acid producing bacteria that have been demonstrated to be viable in the human gastrointestinal tract using the aforementioned in vitro procedure employing the foodstuff substrate as growth medium for the microorganisms.

As a second requirement, the lactic acid producing bacteria of the fermented foodstuff of the present invention are aggregating bacteria, that is the bacteria form aggregates. In addition, or alternatively, the bacteria are capable of co-aggregating with other microorganisms, in particular microorganisms that are pathogenic to humans, that is form aggregates together with the other microorganisms. Preferably, the lactic acid producing bacteria are both aggregating and co-aggregating. The ability to aggregate and/or co-aggregate may be exhibited by the lactic acid producing bacteria under the conditions in the foodstuff substrate during and after fermentation and/or under the conditions prevailing in the human gastrointestinal tract. Preferably, the bacteria are aggregating and/or co-aggregating both in the foodstuff substrate and in the human gastrointestinal tract.

The ability of the microorganisms to aggregate in vitro gives a strong indication of their ability to adhere to the mucus layer in the human gut and to adhere to the epithelial cells of the human intestinal wall and, generally, to colonise the human gastrointestinal tract. This in turn increases the resistance of the person to infection by exclusion of harmful or pathogenic microorganisms from attachment sites. Further, the lactic acid producing bacteria are preferably ones that are coaggregating, that is form coaggregations with other microorganisms, in particular harmful or pathogenic bacteria. In particular, it is preferred that the lactic acid producing bacteria are coaggregating with strains of Salmonella, E. Coli, Clostridium and Methicillin-resistant Staphylococcus aureus (MRSA). This in turn increases the passage and clearance of the harmful bacteria from the intestinal tract of the person consuming the foodstuff.

The ability of a lactic acid producing bacteria to aggregate may be determined by in vitro methods and techniques known in the art, for example as described in Drago, L. et al., noted above, or the method described by Demeckova, V., again noted above. In particular, the bacteria may be cultured in a suitable liquid growth medium, such as Man-Rogosa-Sharpe (MRS) broth (available commercially). Bacterial aggregates may be identified as grains or particles that develop in the liquid culture medium, typically collecting at the bottom of the culture vessel under the action of gravity and leaving a clear supernatant liquid.

Similarly, the ability of the lactic acid producing bacteria to coaggregate with other bacteria may be determined by preparing a co-culture of the lactic acid producing bacteria with one or more target bacteria in like manner with the formation of aggregates being observed as grain-like particles that tend to settle in the culture, again leaving a clear supernatant liquid.

While the formation of aggregates and coaggregates in the bacterial cultures may be observed using the naked eye, as described above, further and more detailed information regarding the aggregating ability of the microorganisms may be obtained by using microscopy techniques, including scanning electron microscopy (SEM).

A procedure for the identification of lactic acid bacteria that are aggregating and coaggregating is set out in Example 2 below.

As a third characteristic, the lactic acid producing bacteria of the fermented foodstuffs of the present invention are capable of producing at least a minimum inhibitory lactic acid concentration in the fermented foodstuff. In respect of the foodstuffs of the present invention, the term ‘minimum inhibitory lactic acid concentration’ is a reference to a lactic acid producing bacteria that is capable of producing at least 150 mMol of lactic acid in 24 hours upon fermentation at 30° C. in a growth medium consisting of MRS broth containing 2% by weight glucose. It has been found that lactic acid producing bacteria that are capable of producing this minimum concentration of lactic acid in the aforementioned test are particularly advantageous in the preparation of fermented foodstuffs for human consumption.

The concentration of lactic acid in the culture medium may be determined using methods known in the art, for example the method of Niven, S. J., et al., The simultaneous determination of short chain fatty acid monosaccharides and ethanol in fermented liquid pig diets', Animal Feed Science and Technology, 117 (2004), (3-4), pages 339 to 345.

A procedure for identifying lactic acid producing bacteria capable of producing at least the minimum inhibitory lactic acid concentration is set out in Example 3 below.

More preferably, the lactic acid producing bacteria is capable of producing at least 200 mMols of lactic acid under the aforementioned procedure and test conditions, still more preferably at least 250 mMols of lactic acid. Lactic acid concentrations of at least 300 mMols, more preferably at least 350 mMols produced under the aforementioned test conditions may also advantageously be applied.

In general, a higher concentration of lactic acid in the fermented foodstuff, and consequently a lower pH value for the foodstuff product is to be preferred. Accordingly, preferably the pH value of the fermented foodstuff is 4.5 or lower, more preferably 4.0 or lower, still more preferably 3.5. The lower limit of pH value and, hence, the upper limit for lactic acid concentration will be, at least in part, determined by the taste of the foodstuff and its acceptability for human consumption.

The lactic acid producing bacteria employed to prepare the fermented foodstuff of the present invention may be either homofermenting or heterofermenting. Heterofermenting bacteria produce lactic acid as a product of their metabolism, along with other organic acids, such as, for example, acetic acid, propionic acid and butyric acid. However, it has been found that the presence of significant quantities of these other acid metabolites may adversely affect the taste of the final product and/or reduce its nutritional value. In contrast, homofermenting lactic acid producing bacteria are ones that metabolise the substrate to produce lactic acid as the only acid metabolite. Accordingly, it is preferred that the lactic acid producing bacteria present in the fermented foodstuff are homofermenting.

Further, the lactic acid producing bacteria used in the fermented foodstuff of the present invention are preferably antagonistic towards pathogens that may cause illness in humans. In particular, it is preferred that the lactic acid producing bacteria have antagonistic activity against one or more strains of Salmonella, E. Coli, Clostridium and Methicillin-resistant Staphylococcus aureus (MRSA).

A procedure for determining the antagonistic activity of a lactic acid producing bacteria is set out in Example 4 below.

In addition, the lactic acid producing bacteria used in the fermented foodstuff of the present invention are preferably capable of adhering to the epithelial cells of the human gastrointestinal tract. In vitro methods for determining the adhesion of bacteria in this manner are known in the art.

Suitable lactic acid producing bacteria for use in the fermented foodstuff of the present invention are naturally occurring and may be isolated from suitable sources using techniques known in the art. Suitable sources of lactic acid producing bacteria for use in the present invention include the gastrointestinal tract of animals and birds. Other sources of lactic acid producing bacteria include cereal grains, spontaneous fermentations in substrates, and the teats and other parts animals. Isolation of the lactic acid producing bacteria may be carried out using techniques known in the art.

Lactic acid producing bacteria may be identified again using techniques known in the art. For example, Lactobacilli may be identified using the gram stain and catalase tests, with Lactobacilli being gram positive and catalase negative rods.

In a further aspect, the present invention provides a method for preparing a fermented foodstuff composition for human consumption, the method comprising fermenting a foodstuff substrate with a lactic acid producing bacteria, the lactic acid bacteria being characterised by:
a) being viable under the conditions prevailing in the human gastrointestinal tract;
b) being an aggregating bacteria and/or co-aggregating with one or more pathogenic bacteria; and
c) being able to produce upon fermentation in the foodstuff substrate lactic acid in an amount of at least a minimum inhibitory concentration of lactic acid.

Still further, the present invention provides the use of a fermented composition as hereindescribed as a foodstuff for humans or in the preparation of a foodstuff for humans.

The fermented foodstuffs of the present invention may be prepared in any suitable manner. Typically, the fermented foodstuffs are prepared by inoculating the foodstuff substrate with an inoculum containing the lactic acid producing bacteria in viable form and fermenting the substrate under suitable conditions. Techniques for fermenting a substrate after inoculation with a lactic acid producing bacteria are known in the art.

The foodstuff composition being fermented contains water. If a dry substrate is being employed, water is added to the substrate. The foodstuff composition being fermented may contain water in an amount of from 1 to 20 parts water by weight for each part of the substrate (dry basis), more preferably from 1 to 15 parts water, by weight. One preferred embodiment comprises the foodstuff substrate and water in a weight ratio of from 1:1 to 1:3, more preferably from 1:1 to 1:2, especially from 1:1 to 1:1.5. In an alternative embodiment, the water content of the foodstuff is higher, with the weight ratio of the foodstuff substrate and water being from 1:5 to 1:20. The finished foodstuff may range from a moist solid to a liquid composition, depending upon the substrate and the water content.

Fermentation of the foodstuff substrate may be conducted at any temperature suitable for the cultivation of the lactic acid producing bacteria. The optimum temperature for fermentation will depend upon the strain or strains of bacteria being employed. Typically, the foodstuff substrate is fermented at a temperature of from 15 to 45° C., more preferably from 30 to 35° C.

The foodstuff substrate is fermented for a sufficient period of time to allow the lactic acid producing bacteria to produce at least a minimum lactic acid concentration of 150 mMol/l lactic acid, more preferably at least 200 mMol/l, still more preferably at least 250 mMol/l. Typical fermentation times are from 8 to 72 hours, more preferably from 8 to 24 hours.

The production of lactic acid in the fermented foodstuff may be monitored by measuring the pH of the foodstuff composition, which will fall as lactic acid is produced during the fermentation process. The pH of the foodstuff composition after fermentation with the lactic acid producing bacteria is preferably 4.5 or lower, more preferably 4.0 or lower.

As noted above, the palatability of foodstuffs having a low pH will be determined by the acid producing the prevailing pH. Thus, a foodstuff with a pH of 3.5 generated by lactic acid will likely be considerably more palatable than a foodstuff having a similar pH arising from acetic, propionic or butyric acid. Fermented foodstuffs having higher concentrations of lactic acid and pH values below 3.5 may be advantageously combined with other materials to produce a final foodstuff composition that is palatable for human consumption, as long as the minimum inhibitory concentration of lactic acid is maintained.

Nutrients and other components essential to the growth of the lactic acid producing bacteria may be added to the foodstuff substrate, as required. Such nutrients and components will be known in the art.

The foodstuff substrate is fermented to produce a concentration of lactic acid producing bacteria in the foodstuff composition that is beneficial to the person consuming the final composition. In particular, the lactic acid bacteria present in the foodstuff composition after fermentation is completed should be viable in sufficient numbers to colonise the human gastrointestinal tract and form viable colonies therein. Preferably, the concentration of lactic acid producing bacteria in the fermented product is at least 10⁶ CFU/ml, more preferably from 10⁷ to 10¹⁰ CFU/ml, still more preferably from 10⁹ to 10¹⁰ CFU/ml.

The foodstuff composition and method of the present invention may employ any suitable lactic acid producing bacteria, with the proviso that the bacterial strain is not harmful to humans. Preferred lactic acid producing bacteria include strains of Lactobacillus and Pediococcus, with strains of Lactobacillus being particularly preferred. Particularly preferred microorganisms of the strain Lactobacillus include strains of Lactobacillus plantarum and Lactobacillus salivarius.

Extensive work has been carried out to isolate a series of strains of Lactobacillus of particular advantage in the preparation of fermented foodstuffs. The strains were isolated by the general method described hereinbefore and using the detailed method described below. Each of the isolated strains exhibited all three of the properties (a) to (c) described above, making them particularly suitable for use in the preparation of a foodstuff composition according to the present invention. Each of the isolated strains has been deposited on 11 Feb., 2009, with the National Collections of Industrial and Marine Bacteria Ltd., Aberdeen, Scotland (hereafter ‘NCIMB’) and accorded the NCIMB accession numbers set out below. The deposits have been made pursuant to and in satisfaction of the requirements of the Budapest Treaty on the International Recognition of the Deposit of Microorganisms for the Purposes of Patent Protection.

Accordingly, in a further aspect, the present invention provides biologically pure cultures of the following microorganisms:

Lactobacillus plantarum, strain number C28, accession number NCIMB 41605;

Lactobacillus salivarius ss. Salivarius, strain number MS3, accession number NCIMB 41606;

Lactobacillus plantarum, strain number MS18, accession number NCIMB 41607;

Lactobacillus plantarum, strain number VD23, accession number NCIMB 41608;

Lactobacillus salivarius ss. Salivarius, strain number MS6, accession number NCIMB 41609; and

Lactobacillus salivarius ss. Salivarius, strain number MS16, accession number NCIMB 41610.

In a further aspect, the present invention provides a composition for the preparation of a fermented foodstuff for human consumption, the composition comprising one or more of the aforementioned microorganisms and a suitable carrier.

Still further, the present invention provides the use of one or more of the aforementioned microorganisms in the preparation of a fermented foodstuff for human consumption.

On a more general note, it has been found that the administration to humans of lactic acid producing bacteria having the following characteristics:
a) being viable under the conditions prevailing in the human gastrointestinal tract;
b) being an aggregating bacteria and/or co-aggregating with one or more pathogens; and
c) being able to produce upon fermentation in the foodstuff substrate lactic acid in an amount of at least a minimum inhibitory concentration of lactic acid;

is generally advantageous for the health and wellbeing when consumed by persons.

Accordingly, in a further aspect, the present invention provides a method for improving the general health of a person, the method comprising administering to the person lactic acid producing bacteria having the following characteristics:
a) being viable under the conditions prevailing in the human gastrointestinal tract;
b) being an aggregating bacteria and/or co-aggregating with one or more pathogens; and
c) being able to produce upon fermentation in the foodstuff substrate lactic acid in an amount of at least a minimum inhibitory concentration of lactic acid.

The preferred lactic acid producing bacteria for general administering to the subject person are as set out above.

The lactic acid producing bacteria are preferably administered to the person as viable microorganisms, preferably in a concentration of at least 10⁶ CFU/ml, more preferably at least 10⁷ CFU/ml, still more preferably in a concentration of at least 10⁹ CFU/ml.

It has been found that the aforementioned lactic acid producing bacteria are effective in combating microorganisms that are harmful to humans. Accordingly, administering the bacteria to a person can improve the general health of the person, in particular increasing their resistance to infection from potentially harmful microorganisms, in particular enteropathogens. This is particularly the case with the young, old and infirm.

Further, the present invention provides a biologically pure culture of a lactic acid producing bacteria having the following characteristics:
a) being viable under the conditions prevailing in the human gastrointestinal tract;
b) being an aggregating bacteria and/or co-aggregating with one or more pathogens; and
c) being able to produce upon fermentation in the foodstuff substrate lactic acid in an amount of at least a minimum inhibitory concentration of lactic acid.

A composition for providing lactic acid bacteria to a person comprises a viable culture of the aforementioned lactic acid producing bacteria having characteristics (a) to (c) and a suitable carrier.

The present invention will be illustrated by way of the following specific examples and by reference to the accompanying figures, in which:

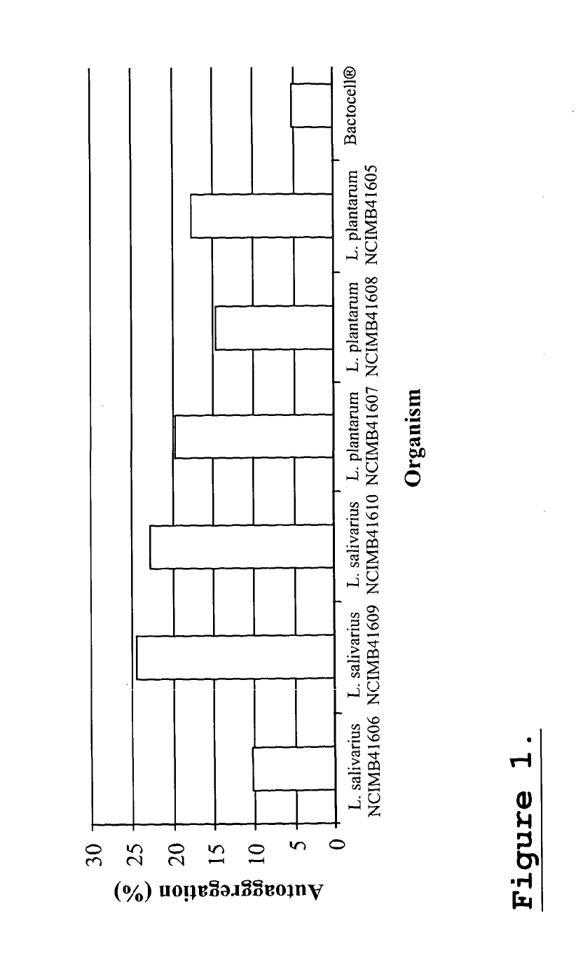

FIG. 1 is a graph of results showing the ability of the lactic acid producing bacteria to autoaggregate following a first test procedure;

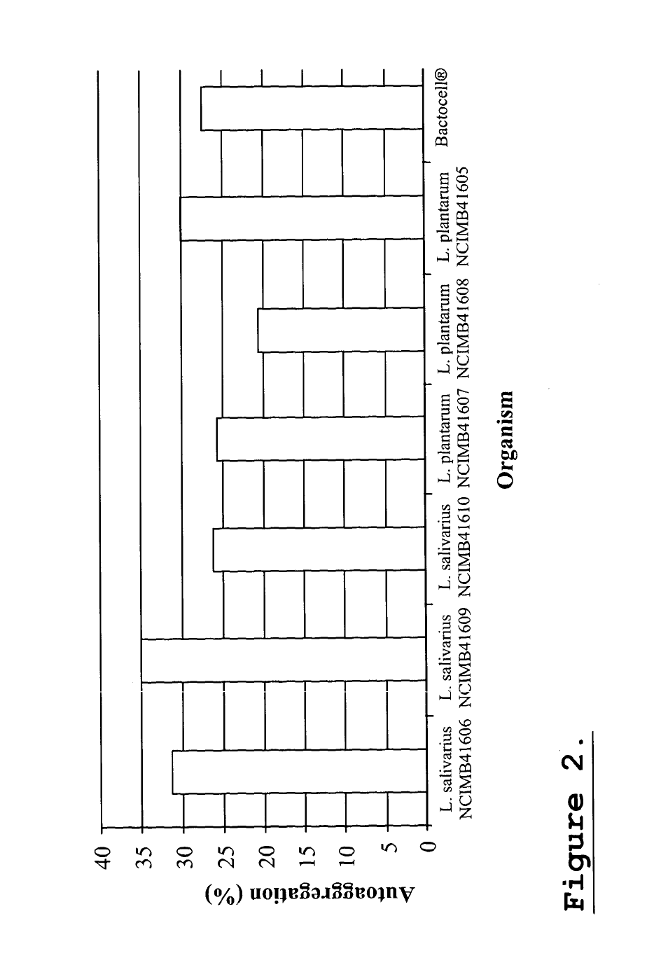

FIG. 2 is a graph of results showing the ability of the lactic acid producing bacteria to autoaggregate following a first second test procedure;

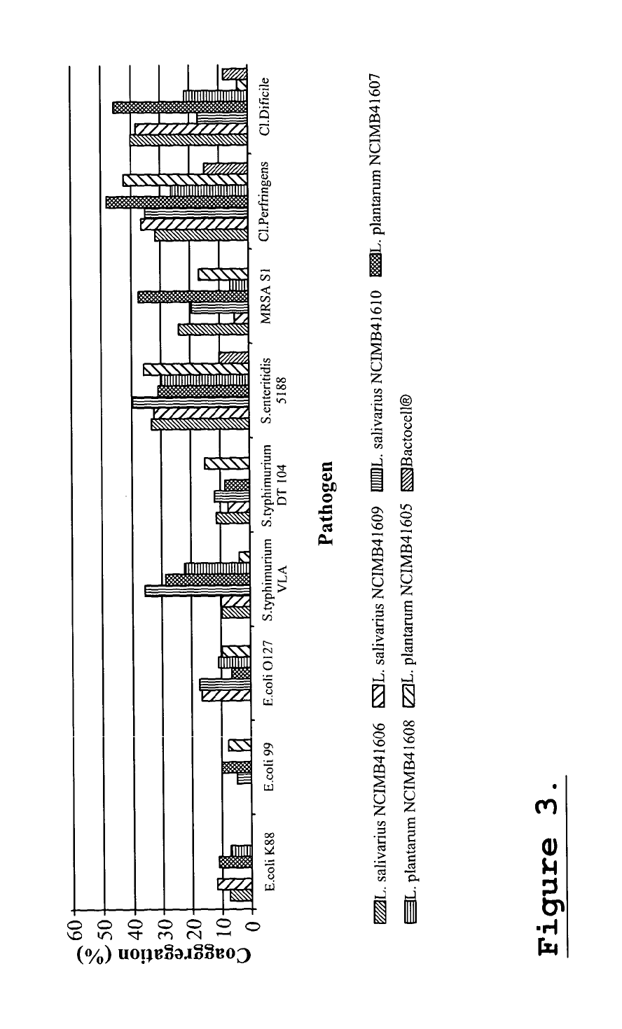

FIG. 3 is a graph of results showing the ability of the lactic acid producing bacteria to coaggregate with the indicated pathogens; and

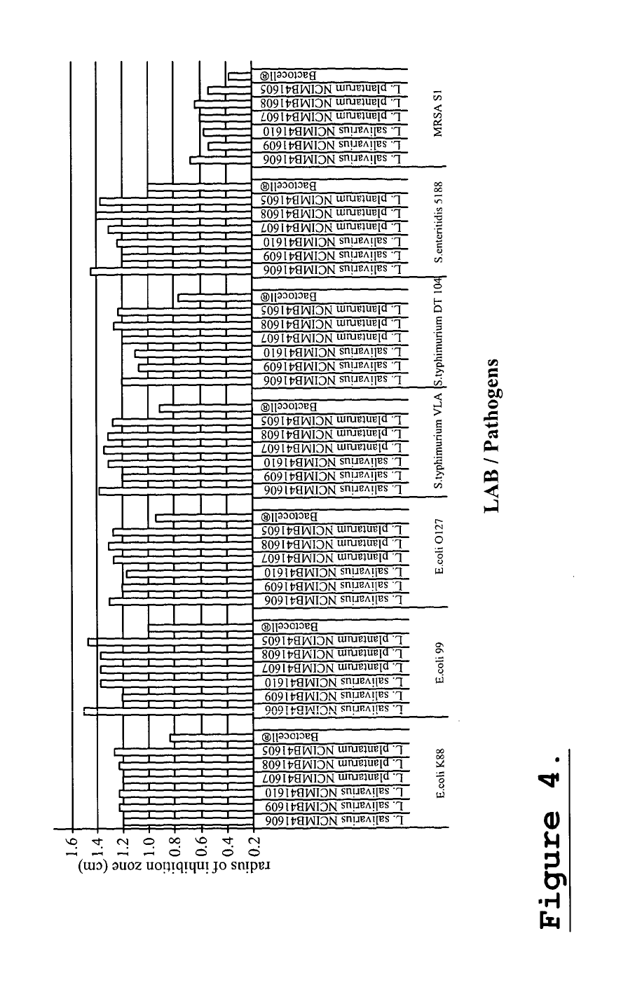

FIG. 4 is a graph of results showing the antagonistic activity of the lactic acid producing bacteria with respect to the indicated pathogens.

The experiments described in the following examples were conducted using lactic acid producing bacteria sourced from pigs and chickens, isolated and identified. The general procedure followed is exemplified by that applied to chickens, as follows:

Three chickens (Hubbard breed; age 9 weeks and 2 days) were fed ad libidum on a diet of a commercially available organic growers ration, grass and clover. The chickens were humanely slaughtered and the entire gastrointestinal tract removed from each bird. Contents from the caecum, jejunum, ileum and crop were removed aseptically. In addition, epithelial cells were removed from the small intestine and the crop by scraping with a slide. All samples were diluted in 10 ml phosphate-buffered saline (PBS, ex. Oxoid, England) and plated in Man-Rogosa-Sharpe (MRS) and Rogosa agar (both ex. Oxoid, England). The streak method of isolation was used to obtain pure cultures from a mixed culture of bacteria. The thus isolated pure cultures were cultured for a second time in MRS agar plates and incubated in anaerobic jars in an atmosphere containing 5% vol carbon dioxide for 72 hours.

A total of 111 lactic acid producing bacteria were isolated on MRS agar (isolation medium for Lactocacillus and Pediococcus strains) and Rogosa agar (isolation medium for Lactobacillus strains).

Gram stains and catalase tests were used to confirm that the isolates were lactic acid producing bacteria. Isolates that were Gram positive and catalase negative were further identified by differential carbohydrate metabolism using API CHL kits (ex. BioMereux, UK).

The lactic acid producing bacteria thus isolated and identified as such were subjected to analysis using the procedures of the following examples to identify those meeting the requirements of:
a) being viable under the conditions prevailing in the human gastrointestinal tract;
b) being an aggregating bacteria and/or co-aggregating with one or more pathogens; and
c) being able to produce upon fermentation in the foodstuff substrate lactic acid in an amount of at least a minimum inhibitory concentration of lactic acid.

In addition, the antagonistic activity of the isolated lactic acid producing bacteria against key pathogenic bacteria was determined.

### EXAMPLES

### Example 1

### Determination of Viability of Lactic Acid Producing Bacteria in the Human Gastrointestinal Tract

The viability of strains of lactic acid producing bacteria in the human gastrointestinal tract may be determined using the following procedure:

Each strain of lactic acid producing bacteria is combined with cows milk. Glucose or sucrose is added to the milk, to provide an energy source for the lactic acid producing bacteria. Alternatively, a carbohydrate source, such as a cereal, may be used.

Prior to combining with the bacteria, the milk is sterilised by irradiation (25 kGy, Con or by heating.

A sample of the inoculated milk is added to a flask, diluted with the addition of distilled water and heated in a water bath to 37° C., to represent the temperature within the human gastrointestinal tract. The pH of the sample of the foodstuff composition is reduced by the addition of HCl (aq; 1M) to adjust the pH in the flask to correspond to the pH found in the human stomach, pH 2.6. The sample is incubated for 90 minutes.

HCl (aq; 1M) is added periodically to each sample throughout the incubation period, in order to maintain the pH at the appropriate level.

Thereafter, sodium hydrogen carbonate (NaHCO₃) is added in sufficient amounts to increase the pH to 8.5, to represent the alkaline conditions in the human intestinal tract. The sample is further incubated for 90 minutes.

Samples (1 ml) of the solution in the flask are removed immediately before the pH was adjusted at each stage in the incubation, diluted with sterile peptone water (9 ml) and 10 fold serial dilutions were prepared. 100 μl of each dilution are spread over MRS agar using aseptic techniques and the plates incubated at 3TC for 24 hours, after which the plates were counted.

The viability of the microorganisms was calculated as the percent of organisms surviving passage through the simulated GI tract.

### Example 2

### Determination of Lactic Acid Bacteria that are Aggregating and Coaggregatinq

The ability of the lactic acid bacteria strains to autoaggregate and form coaggregates with other bacteria was determined using the following procedure:

Seven lactic acid producing bacteria (LAB) were assessed for the ability to autoaggregate and coaggregate with nine pathogens. The lactic acid producing bacteria and their origin are summarised in Table 1 below.

TABLE 1

| Organism—Lactic Acid Bacteria | Origin |
|---|---|
| Lactobacillus salivarius salivarius NCIMB | Chicken, University of |
| 41606 | Plymouth |
| Lactobacillus salivarius salivarius NCIMB | Chicken, University of |
| 41609 | Plymouth |
| Lactobacillus salivarius salivarius NCIMB | Chicken, University of |
| 41610 | Plymouth |
| Lactobacillus plantarum NCIMB 41607 | Chicken, University of |
| | Plymouth |
| Lactobacillus plantarum NCIMB 41608 | Pig, University of Plymouth |
| Lactobacillus plantarum plantarum NCIMB | Pig, University of Plymouth |
| 41605 | |
| Bactocell ®—Pediococcus acidilactici | Commercially available (ex. |
| | Lallemand Animal Nutrition, |
| | France) |

The pathogenic bacteria and their origin are summarised in Table 2 below.

TABLE 2

| Pathogens | Origin |
|---|---|
| Escherichia coli K88 | Seale-Hayne collection (Veterinary Labs |
| | Agency) |
| Escherichia coli K99 | Seale-Hayne collection (Veterinary Labs |
| | Agency) |
| Escherichia coli 0127 | Derriford hospital collection |
| Salmonella typhimurium | Veterinary Laboratory Agency collection |
| Salmonella typhimurium | University of Plymouth collection |
| DT104 | |
| Salmonella enteritidis 5188 | University of Plymouth collection |
| MRSA S1 | University of Plymouth collection |
| Clostridium perfringens | University of Plymouth collection |
| Clostridium dificile | University of Plymouth collection |

Autoaggregation

An autoaggregation assay of the lactic acid producing bacteria listed in Table 1 was performed using two methods.

Following the procedure outlined in Kos, B., et al., ‘Adhesion and aggregation ability of probiotic strain Lactobacillus acidophilus M92’, Journal of Applied Microbiology, 94 (2003), pages 981 to 987, the lactic acid producing bacteria were grown for 18 hours at 37° C. in MRS broth (ex Oxoid) in an atmosphere of 5% vol carbon dioxide. The cells were harvested by centrifugation at 4000 times gravity for 15 minutes, washed twice with phosphate-buffered saline (PBS) and resuspended in PBS to give an optical density (Opposition Division) of 0.5. 4 ml aliquots of each suspension were centrifuged at 4000 times gravity for 15 minutes and the cells resuspended in 4 ml of the filtered sterilised culture supernatant fluid by vortexing for 10 seconds. Autoaggregation was determined after 4 hours of incubation at room temperature by transferring 0.1 ml of the upper suspension to a cuvette containing 0.4 ml of PBS. The absorbance (A) of the sample at 600 nm was measured.

The degree of autoaggregation (%) is expressed as:

1−(A₄/A₀)×100

where A₄ represents the absorbance after incubation for 4 hours and A₀ the absorbance at the start of incubation.

The results are set out in the graph in FIG. 1.

Following the procedure outlined in Del Re, B., et al., ‘Adhesion, autoaggregation and hydrophobicity of 13 strains of Bifidobacterium longum’, Letters in Applied Microbiology, 31 (2000), pages 438 to 442, lactic acid producing bacteria were grown at 37° C. in MRS broth (ex Oxoid) in an atmosphere of 5% vol carbon dioxide for 24 hours. The cells were harvested by centrifugation at 4000 times gravity for 15 min, washed twice with PBS and resuspended in PBS to give an Optical Density (OD) of 0.5. 4 ml aliquots of each bacterial suspension in PBS were centrifuged at 4000 times gravity for 15 min, and the cells were resuspended in 4 mls of the filtered sterilised culture supernatant fluid by vortexing for 10 seconds. After 4 hours of incubation at room temperature, 0.1 ml of the upper suspension was transferred to a cuvette containing 0.4 ml of PBS and the OD was measured at 600 nm. The remaining suspension was mixed by vortexing for 10 seconds and 0.1 ml of the suspension was transferred to a cuvette containing 0.4 ml of PBS to measure the OD of the total bacterial suspension at 600 nm.

The degree of autoaggregation (%) is expressed as:

(1−OD upper suspension/OD total bacterial suspension)×100.

The results are set out in the graph in FIG. 2.

As can be seen in FIGS. 1 and 2, the lactic acid producing bacteria isolated and identified exhibited a very high degree of autoaggregation.

Coaggregation

The ability of the lactic acid producing bacteria to coaggregate with each of the pathogens listed in Table 2 was determined as follows:

The lactic acid producing bacteria were grown at 37° C. in MRS broth (ex Oxoid) in an atmosphere of 5% vol carbon dioxide for 18 hours. Salmonella spp, Escherichia coli and MRSA strains were grown for 18 h aerobically at 37° C. in Nutrient broth (ex Oxoid) and the Clostridium spp strains for 72 h at 37° C. anaerobically in Meat Cooked Medium (ex Oxoid). The cells were harvested by centrifugation at 4000 times gravity for 15 minutes, washed twice with PBS and resuspended in PBS to give an Optical Density (OD) of 0.5. 2 ml aliquots of each pathogen were mixed with 2 ml aliquots of each lactic acid bacterium by vortexing for 10 seconds. Control tubes were set up at the same time, containing 4 ml of each bacterial suspension. The absorbance (A) at 600 nm of each suspension was measured both after initial mixing and after incubation for 5 hours at room temperature, by transferring 0.1 ml of the suspension to a cuvette containing 0.4 ml of PBS.

The degree of coaggregation (%) is calculated using the following equation:

$$Coaggregation ( \% ) = \frac{( \frac{Ax + Ay}{2} ) - A ( x + y )}{( \frac{Ax + Ay}{2} )} \times 100$$

where x and y represent each of the two strains in the control tubes, and (x+y) the mixture.

The results are set out in the graph in FIG. 3.

As can be seen in FIG. 3, the lactic acid producing bacteria isolated and identified exhibited a very high degree of coaggregation with the pathogenic bacteria. It is to be noted that the degree of coaggregation of the bacteria isolated in the present case is significantly higher than that of the commercially available product.

### Example 3

### Determination of Lactic Acid Producing Bacteria Capable of Producing at Least the Minimum Inhibitory Lactic Acid Concentration

The ability of the lactic acid producing bacteria strains to produce lactic acid to at least the minimum inhibitory concentration was determined using the following procedure:

Lactic acid producing strains were grown in MRS broth for 24 hrs at 30° C. Ten ml aliquots of fresh MRS broth were inoculated with 0.1 ml of the 24 hr broth culture and incubated at 30° C. Subsamples of 1.0 ml were taken after 12, 24 and 48 hours for lactic acid analysis by high performance liquid chromatography according to the method of Niven et al. Standard MRS broth contains 2% glucose as a carbohydrate source giving a maximum lactic acid yield of 220 mMol/L.

### Example 4

### Determination of Antagonistic Activity of Lactic Acid Producing Bacteria

The antagonistic activity of the lactic acid producing bacteria strains listed in Table 1 with respect to the pathogenic E. coli, Salmonella and MRSA microorganisms listed in Table 2 was determined using the following procedure:

Antagonistic activity was quantified by the agar spot test described by Jin, L. Z., et al., ‘Antagonistic effects of intestinal Lactobacillus isolates on pathogens of chickens’, Letters in Applied Microbiology, 23 (1996), pages 67 to 71. Following this procedure, cultures of the strains of lactic acid producing bacteria were grown in MRS broth (ex Oxoid) and incubated at 37° C., under anaerobic conditions, for 24 hours. 5 μl aliquots of the cultures of 10⁹ CFU ml⁻¹ were spotted onto the surface of MRS agar plates and incubated, for a further 24 hours, at 5% CO₂ and 37° C., to allow colonies to develop. Approximately 10⁶ CFU ml⁻¹ of each pathogenic bacterium, in 15 ml of nutrient agar kept at 46° C., were poured onto each plate and the plate incubated for a further 24 hours at 37° C. After the incubation the plates were checked for inhibition zones around the lactobacilli spot and the radius of any inhibition zone was recorded. The test of each Lactobacillus strain against each of the pathogens was carried out in triplicates.

The results are set out in the graph in FIG. 4.

As can be seen in FIGS. 1 and 2, the lactic acid producing bacteria isolated and identified exhibited a very high degree of antagonism to the pathogenic bacteria indicated. In particular, it is to be noted that the bacteria of the present invention exhibited antagonism to a significantly higher degree than the commercially available product.

### Example 5

### Fermentation of Milk as a Foodstuff Substrate for Human Consumption

Lactic acid producing bacteria listed in Table 1 were tested for their ability to ferment two milk substrates according to the following procedures:

A commercially available dried skimmed milk powder was prepared according to the manufacturer's instructions to provide a first milk substrate. A milk replacer for calves (70 g) was mixed with water (100 g) to provide a second milk substrate. Both substrates were placed in a steamer for 30 minutes/day for each of 3 days for sterilisation.

Each lactic acid producing bacteria was grown in MRS broth (ex Oxoid) for 24 hours at 37° C.

100 ml of each milk substrate was inoculated with 100 μl of the culture of each lactic acid producing bacteria. The samples were incubated at 30° C. for 48 hours. The ability of the bacteria to ferment the milk substrate was determined by monitoring the pH at intervals during the incubation of 0, 8, 24, 32 and 48 hours, with a reduction in the pH indicating the production of lactic acid. The results for the first and second milk substrates are set out in Tables 3 and 4 respectively.

TABLE 3

| | Time/pH | | | | |
|---|---|---|---|---|---|
| LAB | 0 h | 8 h | 24 h | 32 h | 48 h |
| NCIMB 41606 | 6.69 | 6.61 | 5.55 | 4.76 | 4.35 |
| NCIMB 41609 | 6.69 | 6.62 | 5.72 | 4.98 | 4.56 |
| NCIMB 41610 | 6.69 | 6.68 | 5.69 | 4.96 | 4.46 |
| NCIMB 41607 | 6.69 | 6.62 | 5.83 | 5.08 | 4.53 |
| NCIMB 41608 | 6.69 | 6.65 | 5.85 | 4.96 | 4.45 |
| NCIMB 41605 | 6.69 | 6.65 | 6.09 | 5.22 | 4.60 |

TABLE 4

| | Time/pH | | | | |
|---|---|---|---|---|---|
| LAB | 0 h | 8 h | 24 h | 32 h | 48 h |
| NCIMB 41606 | 5.82 | 6.03 | 6.01 | 5.92 | 5.37 |
| NCIMB 41609 | 5.55 | 5.69 | 5.70 | 5.58 | 4.93 |
| NCIMB 41610 | 5.55 | 5.67 | 5.72 | 5.54 | 4.76 |
| NCIMB 41607 | 5.54 | 5.69 | 5.75 | 5.61 | 4.80 |
| NCIMB 41608 | 6.12 | 6.44 | 6.50 | 6.25 | 5.71 |
| NCIMB 41605 | 6.13 | 6.46 | 6.51 | 5.90 | 5.72 |

The results in Tables 3 and 4 demonstrate that the lactic acid producing bacteria of the present invention are particularly suitable for the preparation of a fermented foodstuff from a substrate, such as a milk substrate.

The foodstuff of the present invention provides particular advantages to the persons consuming the food, especially when the consumer is vulnerable to infection and illness.

In particular, the foodstuff of the present invention provides an advantageous fermented weaning food for infants. In this respect, it is still common practice for infants in less developed countries to be weaned from breast milk onto fermented gruels (usually based on maize or sorghum) fractions. Where these are prepared artisanally in unhygienic conditions and with contaminated water, the foodstuff can be heavily contaminated with enteropathogens, particularly haemolytic E. coli and Salmonella spp. Consequently infant mortality is increased due to infants contracting diarrhoea and dysentery. A weaning food produced by the present invention can improve the health of such infants and significantly reduce mortality.

Further, subjects suffering ill health, immune compromised or with a perturbed GIT function, for example arising from ill-health, medication or old age, can benefit from a fermented food of the present invention, in particular that increases the barrier function of the stomach to enterpopathogens, populates the GIT with beneficial organism that have probiotic properties, and upregulates the person's immune system.

The foodstuff of the present invention also benefits patients with perturbations of the GI tract, for example sufferers of Crohns disease, IBS, and ulcerative colitis, by modulating the gut ecosystem and influencing the immune response.

1-32. (canceled)

33. A fermented foodstuff composition for human consumption, the foodstuff composition being prepared by the fermentation of a foodstuff substrate with a lactic acid producing bacteria, the lactic acid bacteria being characterised by:
a) being viable under the conditions prevailing in the human gastrointestinal tract;
b) being a bacteria capable of aggregating and/or coaggregating with one or more pathogens; and
c) being able to produce upon fermentation in the foodstuff substrate lactic acid in an amount of at least a minimum inhibitory concentration of lactic acid.

34. The fermented foodstuff according to claim 33, wherein the substrate comprises milk, milk fractions, or a carbohydrate-rich food source.

35. The fermented foodstuff according to claim 34, wherein the carbohydrate-rich food source comprises raw or cooked cereals or cereal fractions, potato starch or cassaya.

36. The fermented foodstuff according to claim 35, wherein the raw or cooked cereals or cereal fractions are oats, wheat, barley, maize, millet or sorghum.

37. The fermented foodstuff according to claim 33, wherein the food substrate has a water content of at least 20% by weight.

38. The fermented foodstuff according to claim 33, wherein the ratio of dry food substrate to water is from 1:0.25 to 1:4.

39. The fermented foodstuff according to claim 33, wherein the lactic acid producing bacteria are viable under a pluraltiy of conditions of pH corresponding to a plurality of locations in the human gastrointestinal tract.

40. The fermented foodstuff according to claim 33, wherein the lactic acid producing bacteria are proven viable in an in vitro experiment modelling the conditions in the human gastrointestinal tract, wherein the in vitro experiment employs the food substrate as the growth medium for the lactic acid producing bacteria.

41. The fermented foodstuff according to claim 33, wherein the lactic acid producing bacteria are coaggregating with respect to bacteria that are harmful or pathogens to humans.

42. The fermented foodstuff according to claim 41, wherein the lactic acid producing bacteria are coaggregating with strains of Salmonella, E. Coli, Clostridium and Methicillin-resistant Staphylococcus aureus (MRSA).

43. The fermented foodstuff according to claim 42, wherein the lactic acid producing bacteria are capable of producing at least 275 mMol/l of lactic acid in 48 hours upon fermentation at 30° C. in a growth medium consisting of MRS broth with 2% by weight glucose.

44. The fermented foodstuff according to claim 33, having a pH of 4.5 or lower.

45. The fermented foodstuff according to claim 33, wherein the lactic acid producing bacteria is homofermenting.

46. The fermented foodstuff according to claim 33, wherein the lactic acid producing bacteria is antognistic against one or more pathogens common in the target animal.

47. The fermented foodstuff according to claim 33, wherein the concentration of lactic acid producing bacteria in the fermented foodstuff is at least 10⁶ CFU/ml.

48. The fermented foodstuff according to claim 33, wherein the lactic acid producing bacteria comprise strains of Lactobacillus and/or Pediococcus.

49. The fermented foodstuff according to claim 48, wherein the lactic acid producing bacteria are selected from one or more of:
Lactobacillus plantarum, strain number C28, accession number NCIMB 41605;
Lactobacillus salivarius ss. Salivarius, strain number MS3, accession number NCIMB 41606;
Lactobacillus plantarum, strain number MS18, accession number NCIMB 41607;
Lactobacillus plantarum, strain number VD23, accession number NCIMB 41608;
Lactobacillus salivarius ss. Salivarius, strain number MS6, accession number NCIMB 41609; and
Lactobacillus salivarius ss. Salivarius, strain number MS16, accession number NCIMB 41610.

50. An inoculant for the preparation of a fermented human foodstuff from a foodstuff substrate, the inoculant comprising a viable culture of a lactic acid producing bacteria having the following characteristics:
a) being viable under the conditions prevailing in the human gastrointestinal tract;
b) being a bacteria capable of aggregating and/or co-aggregating with one or more pathogens; and
c) being able to produce upon fermentation in the foodstuff substrate lactic acid in an amount of at least a minimum inhibitory concentration of lactic acid.

51. The inoculant according to claim 50, wherein the lactic acid producing bacteria are selected from one or more of:
Lactobacillus plantarum, strain number C28, accession number NCIMB 41605;
Lactobacillus salivarius ss. Salivarius, strain number MS3, accession number NCIMB 41606;
Lactobacillus plantarum, strain number MS18, accession number NCIMB 41607;
Lactobacillus plantarum, strain number VD23, accession number NCIMB 41608;
Lactobacillus salivarius ss. Salivarius, strain number MS6, accession number NCIMB 41609; and
Lactobacillus salivarius ss. Salivarius, strain number MS16, accession number NCIMB 41610.

52. A method for preparing a fermented foodstuff composition for human consumption, the method comprising fermenting a foodstuff substrate with a lactic acid producing bacteria, the lactic acid bacteria being characterised by:
a) being viable under the conditions prevailing in the human gastrointestinal tract;
b) being an aggregating bacteria and/or co-aggregating with one or more pathogenic bacteria; and
c) being able to produce upon fermentation in the foodstuff substrate lactic acid in an amount of at least a minimum inhibitory concentration of lactic acid.

53. The method according to claim 52, wherein the lactic acid producing bacteria are selected from one or more of:
Lactobacillus plantarum, strain number C28, accession number NCIMB 41605;
Lactobacillus salivarius ss. Salivarius, strain number MS3, accession number NCIMB 41606;
Lactobacillus plantarum, strain number MS18, accession number NCIMB 41607;
Lactobacillus plantarum, strain number VD23, accession number NCIMB 41608;
Lactobacillus salivarius ss. Salivarius, strain number MS6, accession number NCIMB 41609; and
Lactobacillus salivarius ss. Salivarius, strain number MS16, accession number NCIMB 41610.
